(19) United States
(12) Patent Application Publication
(10) Pub. No.: US 2013/0164386 A1
(43) Pub. Date: Jun. 27, 2013

(54) Targeting the Alpha3 Subnit of Na+-K+-ATPase for the Treatment of Cough with Sulfenamides

(71) Applicant: Brendan J. Canning, Ellicott City, MD (US)

(72) Inventor: Brendan J. Canning, Ellicott City, MD (US)

(73) Assignee: THE JOHNS HOPKINS UNIVERSITY, Baltimore, MD (US)

(21) Appl. No.: 13/704,722

(22) PCT Filed: Jun. 17, 2011
(86) PCT No.: PCT/US11/40817

§ 371 Date: Jan. 16, 2013

Related U.S. Application Data
(60) Provisional application No. 61/355,753, filed on Jun. 17, 2010.

(51) Int. Cl. A61K 31/4439; A61K 45/06

(52) U.S. Cl. CPC A61K 31/4439; A61K 45/06
USPC 424/642; 435/18; 514/338; 514/222.8

(57) ABSTRACT

Therapeutic agents that target the alpha3 expressing isozymes of the sodium pump at the peripheral terminals of cough receptors and methods of use thereof to prevent or treat cough are disclosed.

### CROSS-REFERENCE TO RELATED APPLICATION

This application claims the benefit of U.S. Provisional Application No. 61/355,753, filed Jun. 17, 2010, which is incorporated herein by reference in its entirety.

### FEDERALLY SPONSORED RESEARCH OR DEVELOPMENT

This invention was made in part with United States Government support under NHLBI R01 HL083192 awarded by the National Institutes of Health (NIH). The U.S. Government has certain rights in the invention.

### BACKGROUND

Cough is the most common presenting symptom amongst patients seeking advice from primary healthcare providers. Chronic cough adversely impacts patient quality of life, and in the acute setting, cough is a major route of pathogen spread. Current antitussive therapies are minimally effective and have side effects that limit their utility. In the United States alone, over two billion dollars are spent annually on over-the-counter cough remedies with debatable efficacy and potential toxicity, and billions more are spent on doctor's visits and lost in sick days annually. While little evidence exists to suggest that cough protects patients from infections, it is widely held that cough is a primary mechanism of transmitting deadly infections, including all forms of influenza, tuberculosis, and Bordetella pertussis, the gram-negative bacteria causing whooping cough. Cough thus represents a major public health issue that is poorly treated with existing strategies and therapeutics.

Recent research has identified the vagal afferent nerve subtypes essential for the initiation of the cough reflex. These vagal afferent nerves, or “cough receptors,” are readily differentiated from other vagal afferent nerves based on several physiological and morphological properties. More recently, studies have identified the unique expression of the alpha3 subunit of Na⁺—K⁺-ATPase at the peripheral terminals of these vagal afferent nerves. Subsequent electrophysiological and functional studies suggest that isozymes of the sodium pump utilizing the alpha3 subunit may play an essential role in the initiation of cough. A therapeutic agent that selectively targets the activation of these cough receptors could have beneficial effects in treating acute and chronic forms of cough.

### SUMMARY

In some aspects, the presently disclosed subject matter provides a method for treating or preventing an acute or chronic cough in a subject in need of treatment thereof by modulating an activity of an alpha3 subunit of Na⁺K⁺-ATPase on one or more vagal afferent nerves of the subject, the method comprising: (a) converting an acid-labile sulfenamide prodrug to a sulfenamide; and (b) topically administering, e.g., in some aspects, as a aerosol to the subject's airway, a therapeutically effective amount of the sulfenamide to the subject; whereby the sulfenamide modulates the activity of an alpha3 subunit of Na⁺K⁺-ATPase on one or more vagal afferent nerves of the subject to treat or prevent the cough.

In particular aspects, the acid-labile sulfenamide prodrug is selected from the group consisting of a benzimidazole and an imidazopyridine, and derivatives, enantiomers, isomers, tautomers, free bases, polymorphs, esters, hydrates, or salts thereof. In more particular aspects, the benzimidazole or imidazopyridine comprises a compound of formula (I):

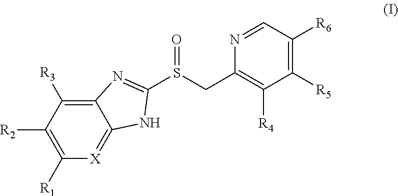

wherein: X is CH or N; R₁, R₂, and R₃ are the same or different and are selected from the group consisting of hydrogen, substituted and unsubstituted alkyl, alkoxyl, halogen, haloalkoxyl, alkylcarbonyl, alkoxycarbonyl, oxazolinyl, trifluoroalkyl, a heterocyclic ring that may be further substituted or adjacent groups R₁, R₂, and R₃ form ring structures, which may be further substituted; R₄, R₅, and R₆ are the same or different and are selected from the group consisting of hydrogen, substituted or unsubstituted alkyl, alkoxyl optionally substituted by halogen, alkylthio, alkoxyalkoxyl, dialkylamino, piperidino, morpholino, halogen, phenyl and phenylalkoxyl; or a derivative, enantiomer, isomer, tautomer, free base, polymoph, ester, hydrate, or salt thereof.

In some aspects, the benzimidazole or imidazopyridine is selected from the group consisting of omeprazole, esomeprazole, hydroxyomeprazole, rabeprazole, pantoprazole, lansoprazole, s-lansoprazole, dexlansoprazole, pariprazole, ilaprazole, dontoprazole, habeprazole, perprazole, ransoprazole, nepaprazole, leminoprazole, tenatoprazole, and s-tenatoprazole.

In other aspects, the presently disclosed methods further comprise administering one or more zinc supplements in combination with the presently disclosed sulfenamides.

In yet other aspects, the presently disclosed subject matter provides a method for identifying at least one candidate compound for treating or preventing a cough in a subject by modulating an activity of an alpha3 subunit of Na⁺K⁺ATPase on one or more vagal afferent nerves of the subject, the method comprising: (a) obtaining a sample containing a functional alpha3 subunit of Na+K+ATPase on a vagal afferent nerve; (b) contacting the sample with an amount of a candidate compound sufficient to modulate the activity of the alpha3 subunit of Na⁺K⁺ATPase on the vagal afferent nerve; (c) measuring the activity of the alpha3 subunit of Na⁺K⁺ATPase on the vagal afferent nerve; and (d) comparing the activity of the alpha3 subunit of Na⁺K⁺ATPase on the vagal afferent nerve that was contacted with the candidate compound with the activity an alpha3 subunit of Na⁺K⁺ATPase on the vagal afferent nerve that was not contacted with the candidate compound, wherein a modulation of the activity of the alpha3 subunit of Na⁺K⁺ATPase on the vagal afferent nerve by the candidate compound identifies the compound as a candidate to treat or prevent a cough.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIGS. 1A-1D show: (A) selective immunohistochemical labeling of cough receptor terminations in the guinea pig tracheal mucosa with antisera to the alpha3 subunit of Na+K+-ATPase; (B) the cough receptor terminals innerating the tracheal mucosa also are brilliantly and selectively labeled intravitally by the styryl dye FM2-10. This staining method depends upon active Na+-K+ ATPase. (C) the Na+-K+ ATPase inhibitor ouabain nearly abolished citric acid evoked coughing in anesthetized guinea pigs at a dose (30 μg/kg lv) that had no effect on heart rate, blood pressure or respiratory pattern at eupnea; and (D) topically applied, acid activated omeprazole (0.1 mM) nearly abolished citric acid evoked coughing in anesthetized guinea pigs; and

FIG. 2 demonstrates that acid-activated omeprazole (1 mg/mL), delivered as an aerosol to the airways, markedly inhibits citric acid evoked coughing in conscious (unanesthetized) guinea pigs.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Role of the Alpha3 Subunit of Na⁺—K⁺-ATPase in Regulating Cough

Cough is the most commonly reported presenting symptom amongst patients seeking medical advice. In many patients, cough persists despite aggressive medical treatment of their underlying illnesses. Cough adversely impacts quality of life, but, unfortunately, existing antitussives are nonspecific, minimally effective, and often have undesirable side effects that limit their utility. In the United States alone, over two billion dollars are spent annually on over-the-counter cough remedies with debatable efficacy and potential toxicity, and billions more are spent annually on sick days and doctor's visits. While little evidence exists to suggest that cough protects the human population from infections, it is widely held that cough is a primary mechanism of transmission of deadly infections, including all forms of influenza, tuberculosis, and Bordetella pertussis, the gram-negative bacteria causing whooping cough. Cough thus represents a major public health issue that is poorly treated with existing therapeutic agents and strategies.

The current understanding of the pharmacology, physiology, and pathophysiology of cough is derived primarily from studies done in animals. Recent research has defined the vagal afferent nerve subtypes regulating the cough reflex. One afferent nerve subtype of particular interest plays an essential role in the initiation of cough. These “cough receptors” are differentiated from other airway sensory nerves by several physiological and pharmacological attributes. Therapeutic agents that selectively target the activation of the cough receptors could have beneficial effects in treating acute and chronic forms of cough.

More recent studies have shown that the cough receptors uniquely express the alpha3 subunit of Na⁺K⁺-ATPase in their peripheral terminals. Na⁺K⁺-ATPases, or the sodium pumps, play an essential role in all cells in maintaining membrane Na⁺-gradients, a function essential for life that accounts for as much as 40% of all energy consumption in the body. Sodium pumps move sodium ions out of cells in exchange for potassium. Most of the sodium pumping function in all cells is attributed to “housekeeping” sodium pump isozymes, utilizing an alpha1 subunit, one of three beta subunits and modulatory gamma subunits (or FXYD proteins). Subunit composition varies amongst organs and cell types, but all cells likely express and rely mostly on the alpha1 subunit for maintaining Na⁺ gradients.

The alpha3 subunit is found primarily in neurons. Despite knowledge of its existence, and several detailed studies of its physiological and pharmacological properties, it has been difficult to attribute a specific function to the alpha3 subunit of the sodium pump. This subunit of Na⁺K⁺-ATPase has a high affinity for ATP (which, upon hydrolysis, drives pump function), but a comparatively low affinity for sodium. These characteristics suggest that the alpha3 isozyme may become important in highly active cells in which energy (ATP) usage is excessive and Na⁺ influx and accumulation increases. Such a role has been proposed for the alpha3 subunit in regulating transmission of high frequency inputs associated with hearing.

In studies of the cough reflex, it has been found that sustained high frequency activation of the cough receptors is required to reach threshold for cough initiation. Immunohistochemical studies revealed that the cough receptors uniquely express the alpha3 subunit of the Na⁺-pump in their peripheral terminals of the cough receptors and are intensely and selectively labeled intravitally by the styryl dye FM2-10. This labeling was not attributable to vesicular uptake of the dye by the terminals or permeation through ion channels.

Previous studies suggested that styryl dye fluorescence occurs in some cells secondary to sodium pump function. Consistent with this suggestion, it has been shown that FM2-10 labeling of the cough receptors could be prevented by sodium pump inhibition with the endogenous steroid ouabain. Mazzone, S. B, et al., “Selective Expression of a Sodium Pump Isozyme by Cough Receptors and Evidence for Its Essential Role in Regulating Cough,” J. Neurosci., 2009, 29(43):13662-13671. Thus, the cough receptors uniquely express the alpha3 subunit of the sodium pump in their peripheral terminals, and these terminals display high basal sodium pump activity based on FM2-10 fluorescence. Subsequent electrophysiological and functional studies showed that high frequency action potential patterning in cough receptors in vitro, and coughing evoked in vivo, were reduced substantially by administering an alpha3-selective concentration of the Na+-K+ ATPase inhibitor ouabain. Indeed, at an intravenous dose that had no effect on heart rate, blood pressure or respiration, ouabain nearly abolished citric acid evoked coughing in guinea pigs.

In summary, to date, researchers have defined the vagal afferent nerves regulating cough, shown that the cough receptors uniquely express the alpha3 subunit of Na⁺—K⁺-ATPase in their peripheral terminals, demonstrated inhibition of the alpha3 subunit of Na+-K+-ATPase in their peripheral terminals, and shown that inhibition of the alpha3 expressing isozyme of ATPase profoundly and selectively inhibits cough receptor excitability and coughing. Ouabain might thus be used clinically to treat cough. But, ouabain is not very selective for the alpha3 expressing isozymes of the sodium pump, and can be toxic at high doses with worrisome side effects due to its ability to inhibit housekeeping sodium pump function. Accordingly, more potent and selective inhibitors of the alpha3 subunit would be preferable.

### II. Method and Therapeutic Agents for Treating or Preventing Cough by Modulating the Activity of an Alpha3 Subunit of Na⁺K⁺-ATPase the Vagal Afferent Nerves

In some embodiments, the presently disclosed subject matter provides a method for treating or preventing a cough in a subject in need of treatment thereof by modulating an activity of an alpha3 subunit of Na⁺K⁺-ATPase on one or more vagal afferent nerves of the subject, the method comprising: (a) converting an acid-labile sulfenamide prodrug to a sulfenamide; and (b) topically administering a therapeutically effective amount of the sulfenamide to the subject; whereby the sulfenamide modulates the activity of an alpha3 subunit of Na⁺K⁺-ATPase on one or more vagal afferent nerves of the subject to treat or prevent the cough.

As used herein the term “acid labile sulfenamide prodrug” refers to a compound, which, when contacted or exposed to an acid is converted to a sulfenamide, i.e., a compound of the general formula —RSNR₂. Accordingly, in some embodiments, the converting of the acid-labile sulfenamide prodrug to a sulfenamide comprises contacting the prodrug with an acid. Methods of converting the acid labile sulfenamide prodrug to a sulfenamide are known in the art and include dissolving the prodrug in an acidic solution, which in some embodiments, can have a pH of about 3 to about 4. See Keeling D. J., Fallowfield C., Milliner K. J., Tingley S. K., Ife R. J., Underwood A. H., “Studies on the mechanism of action of omeprazole,” Biochem Pharmacol. 1985; 34(16):2967-73. In some embodiments, the sulfenamide is stable and can be stored and administered at a later time, whereas in other embodiments, the prodrug is converted to the sulfenamide prior to, e.g., in some embodiments, immediately before, administration.

In particular embodiments, the acid-labile sulfenamide prodrug is selected from the group consisting of a benzimidazole and an imidazopyridine, and derivatives, enantiomers, isomers, tautomers, free bases, polymorphs, esters, hydrates, or salts thereof.

Benzimidazoles are used clinically to treat gastroesophageal reflux disease based largely on their ability to inhibit gastric parietal cell proton pumps, the H⁺K⁺-ATPase, comprised of subunits that share some homology with sodium pump isozyme subunits. Further, benzimidazoles, such as omeprazole and lansoprazole, are prodrugs. Upon ingestion, the benzimidazoles are converted to sulfenamides by the acid in the stomach.

Sulfenamides have a highly reactive sulfur moiety that can interact covalently with sulfur groups in proteins, or more specifically, the sulfhydryl groups of the amino acid cysteine. Such covalent linkage accounts for the ability of the benzimidazoles to inhibit the parietal cell proton pump. Once “acid activated,” however, the sulfenamides have the potential to become promiscuous agents, covalently linking with cysteines in any protein not sterically hindered in access for the sulfenamides. Indeed, not long after the discovery of the proton pump inhibitors, several studies detailed the ability of acid-activated sulfenamides to interact with and inhibit an isozyme of Na+-K+-ATPase. These discoveries were made long before all sodium pump subunits had been cloned and identified and have gone largely ignored in the 20 years since their original description.

As provided herein above, the alpha3 subunit of the sodium pump plays an important role in regulating cough. Previous studies of the proton pump inhibitors lansoprazole and omeprozole have shown that these drugs may selectively interact with the “brain-specific” isozyme of the sodium pump (likely the alpha3 subunit expressing isozymes). The presently disclosed subject matter demonstrates that sulfenamides are potent and selective inhibitors of the alpha3 expressing isozymes of the sodium pump, and are effective antitussive agents.

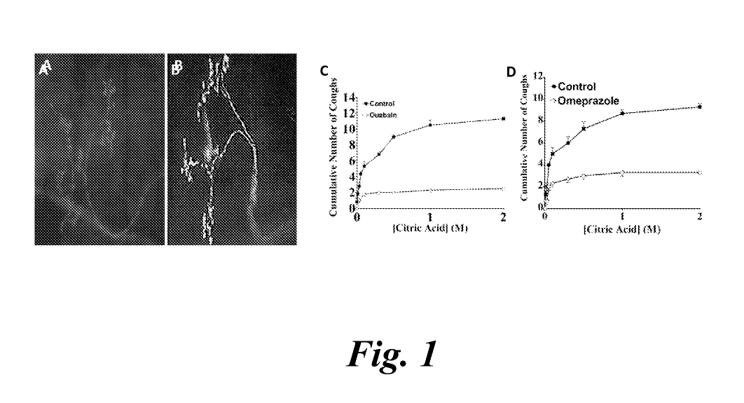

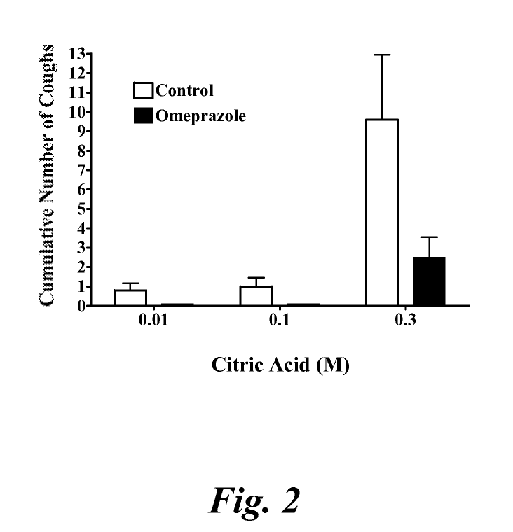

In particular embodiments, the presently disclosed subject matter demonstrates that omeprazole applied topically to the tracheal mucosa nearly abolishes acid evoked coughing in anesthetized guinea pigs (see FIG. 1). In further embodiments, the presently disclosed subject matter demonstrates that nebulized acid-activated omeprazole inhibits acid evoked coughing in conscious guinea pigs (see FIG. 2).

Gastroesophageal reflux disease is widely believed to be a major cause of chronic cough. The evidence in support of this belief is partly the well-documented evidence of reflux symptoms in coughers and objective evidence of acid reflux in coughers based on ambulatory pH monitoring. But, the most frequently cited evidence that reflux is a cause of chronic cough is that proton pump inhibitors like omeprazole, when used continuously, reduce coughing in patients even in patients that show no other signs or symptoms of reflux disease. Interestingly, H2 blockers, which for decades were the only effective treatment for GERD and are still widely used to treat reflux diseases, have no effect on cough.

Even the data with the proton pump inhibitors are intriguing. Proton pump inhibitors will reduce stomach acidity within minutes of ingestion and remain effective at neutralizing gastric fluid (by inhibiting acid secretion) for as long as they are ingested. But, it takes weeks of treatment to realize any benefit from proton pump inhibition to produce any effect on cough symptoms. Without wishing to be bound to any one particular theory, it is thought that acid activation and covalent linkage to the alpha3 subunit of the sodium pump within the airways and on the sensory nerve terminals that regulate cough accounts for the antitussive effects of the benzimidazoles. Studies in experimental animals and in human subjects challenged with citric acid to initiate coughing are supportive of this assertion.

In more particular embodiments, the benzimidazole or imidazopyridine prodrug comprises a compound of formula (I):

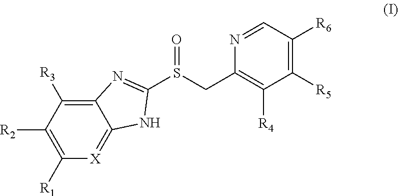

wherein: X is CH or N; R₁, R₂, and R₃ are the same or different and are selected from the group consisting of hydrogen, substituted and unsubstituted alkyl, alkoxyl, halogen, haloalkoxyl, alkylcarbonyl, alkoxycarbonyl, oxazolinyl, trifluoroalkyl, a heterocyclic ring that may be further substituted or adjacent groups R₁, R₂, and R₃ form ring structures, which may be further substituted; R₄, R₅, and R₆ are the same or different and are selected from the group consisting of hydrogen, substituted or unsubstituted alkyl, alkoxyl optionally substituted by halogen, alkylthio, alkoxyalkoxyl, dialkylamino, piperidino, morpholino, halogen, phenyl and phenylalkoxyl; and derivatives, enantiomers, isomers, tautomers, free bases, polymophs, esters, hydrates, or salts thereof.

In particular embodiments, the benzimidazole or imidazopyridine is selected from the group consisting of omeprazole, esomeprazole, hydroxyomeprazole, rabeprazole, pantoprazole, lansoprazole, s-lansoprazole, dexlansoprazole, pariprazole, ilaprazole, dontoprazole, habeprazole, perprazole, ransoprazole, nepaprazole, leminoprazole, tenatoprazole, and s-tenatoprazole.

Compounds suitable for use with the presently disclosed subject matter are disclosed in U.S. Pat. No. 6,730,685, for “Formulation of Substituted Benzimidazoles,” to Brill's, issued May 4, 2004, and U.S. Patent Publication No. 2008/0103169 for “Compositions Comprising Acid Labile Proton Pump Inhibiting Agents, at Least One Other Pharmaceutically Active Agent and Methods of Using Same” to Phillips et al., published May 1, 2008, each of which is incorporated herein by reference in its entirety.

One of ordinary skill in the art would appreciate that analogs and derivatives of the disclosed and related agents also are suitable for use with the presently disclosed methods. As used herein, an “analog” refers to a chemical compound in which one or more individual atoms or functional groups of a parent compound have been replaced, either with a different atom or with a different functional group. For example, thiophene is an analog of furan, in which the oxygen atom of the five-membered ring is replaced by a sulfur atom.

As used herein, a “derivative” refers to a chemical compound which is derived from or obtained from a parent compound and contains essential elements of the parent compound but typically has one or more different functional groups. Such functional groups can be added to a parent compound, for example, to improve the molecule's solubility, absorption, biological half life, and the like, or to decrease the toxicity of the molecule, eliminate or attenuate any undesirable side effect of the molecule, and the like. An example of a derivative is an ester or amide of a parent compound having a carboxylic acid functional group.

In embodiments wherein the benzimidazole or imidazopyridine is a compound of Formula (I), the sulfenamide has the following formula:

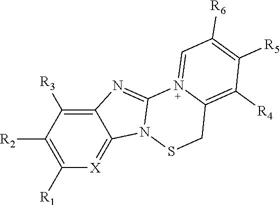

wherein R₁, R₂, R₃, R₄, R₅, R₆, and R₇ are as defined hereinabove.

In certain embodiments, the sulfenamide is topically administered to the subject in an aerosol form. In particular embodiments, the sulfenamide is topically administered to an airway of the subject. To be effective against cough, it is preferable that the presently disclosed sulfenamides reach the lower airways of the subject, such as the larynx, trachea, and bronchi.

In other embodiments, the presently disclosed methods further comprise administering one or more zinc (Zn++) supplements in combination with the presently disclosed sulfenamides. Zinc supplements suitable for use with the presently disclosed methods include, but are not limited to, zinc gluconate, zinc sulfate, zinc acetate, and zinc citrate.

The cough is selected from the group consisting of an acute cough and a chronic cough. As used herein, the term “chronic cough” refers to a cough that has been present for at least eight (8) weeks or longer. An “acute cough” refers to a cough that has been present for less than three (3) weeks.

Accordingly, the presently disclosed subject matter demonstrates that sulfenamides, by virtue of their inhibitory effects on the alpha3 expressing isozyme of the sodium pump, could be broad spectrum antitussives. Such compounds may be effective and novel therapeutics for the treatment of acute and chronic presentations of cough.

### III. Methods for Screening for Candidate Compounds Capable of Modulating an Activity of an Alpha3 Subunit of Na⁺K⁺ATPase

In some embodiments, the presently disclosed subject matter provides a method for identifying at least one candidate compound for treating or preventing a cough in a subject by modulating an activity of an alpha3 subunit of Na⁺K⁺ATPase on one or more vagal afferent nerves of the subject, the method comprising: (a) obtaining a sample containing a functional alpha3 subunit of Na+K+ATPase on a vagal afferent nerve; (b) contacting the sample with an amount of a candidate compound sufficient to modulate the activity of the alpha3 subunit of Na⁺K⁺ATPase on the vagal afferent nerve; (c) measuring the activity of the alpha3 subunit of Na⁺K⁺ATPase on the vagal afferent nerve; and (d) comparing the activity of the alpha3 subunit of Na⁺K⁺ATPase on the vagal afferent nerve that was contacted with the candidate compound with the activity an alpha3 subunit of Na⁺K⁺ATPase on the vagal afferent nerve that was not contacted with the candidate compound, wherein a modulation of the activity of the alpha3 subunit of Na⁺K⁺ATPase on the vagal afferent nerve by the candidate compound identifies the compound as a candidate to treat or prevent a cough.

In some embodiments, the candidate compound is activated with an acid before contacting it with the alpha3 subunit of Na⁺K⁺ATPase on the vagal afferent nerve. In some embodiments, the candidate compound is selected from the group consisting of a benzimidazole and an imidazopyridine, and derivatives, enantiomers, isomers, tautomers, free bases, polymorphs, esters, hydrates, or salts thereof.

### IV. Pharmaceutical Compositions and Administration

In some embodiments, the present disclosure provides a pharmaceutical composition including one or more compounds of Formula (I) alone or in combination with one or more additional therapeutic agents in admixture with a pharmaceutically acceptable excipient. One of skill in the art will recognize that the pharmaceutical compositions include the pharmaceutically acceptable salts of the compounds described above.

In therapeutic and/or diagnostic applications, the compounds of the disclosure can be formulated for a variety of modes of administration, which in preferred embodiments includes topical administration. Techniques and formulations generally may be found in Remington: The Science and Practice of Pharmacy (20^{th} ed.) Lippincott, Williams & Wilkins (2000). In particular embodiments, the presently disclosed sulfenamides are formulated to be administered topically as an aerosol.

The compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). Pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

Depending on the specific conditions being treated, such agents may be formulated into liquid, solid, or aerosol dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Use of pharmaceutically acceptable inert carriers to formulate the compounds herein disclosed for the practice of the disclosure into dosages suitable for topical administration is within the scope of the disclosure. For nasal or inhalation delivery into one or more airways of the subject, the agents of the disclosure also may be formulated by methods known to those of skill in the art, and may include, for example, but not limited to, examples of solubilizing, diluting, or dispersing substances such as, saline, preservatives, such as benzyl alcohol, absorption promoters, and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein.

An “effective amount” of an agent refers to the amount of the agent sufficient to elicit a desired biological response. As will be appreciated by one of ordinary skill in the art, the absolute amount of a particular agent that is effective can vary depending on such factors as the desired biological endpoint, the agent to be delivered, the target cell or tissue, and the like. One of ordinary skill in the art will further understand that an effective amount can be administered in a single dose, or can be achieved by administration of multiple doses.

In addition to the active ingredients, these pharmaceutical compositions may contain suitable pharmaceutically acceptable carriers comprising excipients and auxiliaries that facilitate processing of the active compounds into preparations which can be used pharmaceutically.

Pharmaceutical preparations can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Depending upon the particular condition, or disease state, to be treated or prevented, additional therapeutic agents, which are normally administered to treat or prevent that condition, may be administered together with the compounds of this disclosure. These additional agents may be administered separately, as part of a multiple dosage regimen, from the composition. Alternatively, these agents may be part of a single dosage form, mixed together with the compound in a single composition.

In some embodiments, one or more additional therapeutic agents when administered with the presently disclosed compounds provide a synergistic therapeutic effect. A “synergistic therapeutic effect” refers to a therapeutic effect observed with a combination of two or more therapies and/or therapeutic agents wherein the therapeutic effect (as measured by any of a number of parameters) is greater the sum of the respective individual therapies. Such a synergistic effect may permit a reduction in the dosages of these agents and/or an improvement of the clinical outcome of the subject being treated. Further, a reduced dose of any one particular therapeutic agent may, in turn, reduce one or more unwanted side effects associated with that agent.

Accordingly, in some embodiments, the therapeutically effective amount of a first therapeutic agent and a second therapeutic agent comprises a synergistically effective amount of the first therapeutic agent and the second therapeutic agent. For example, in some embodiments, the first therapeutic agent can be a sulfenamide (or an acid labile sulfenamide prodrug) and the second therapeutic agent can be a zinc supplement.

### V. Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to compounds of Formula (I) are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted, for example, with fluorine at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, iso-propyl, cyclopropyl, allyl, vinyl, n-butyl, tert-butyl, ethynyl, cyclohexyl, methoxy, diethylamino, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁-C₁₀ means one to ten carbons). In particular embodiments, the term “alkyl” refers to C₁₋₂₀ inclusive, linear (i.e., “straight-chain”), branched, or cyclic, saturated or at least partially and in some cases fully unsaturated (i.e., alkenyl and alkynyl)hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, iso-pentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof.

“Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “heteroalkyl,” by itself or in combination with another term, means, unless otherwise stated, a stable straight or branched chain, or cyclic hydrocarbon group, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂₅—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, 0-CH₃, -0-CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)R′, —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups that are limited to hydrocarbon groups are termed “homoalkyl.”

More particularly, the term “alkenyl” as used herein refers to a monovalent group derived from a C₁₋₂₀ inclusive straight or branched hydrocarbon moiety having at least one carbon-carbon double bond by the removal of a single hydrogen atom. Alkenyl groups include, for example, ethenyl (i.e., vinyl), propenyl, butenyl, 1-methyl-2-buten-1-yl, pentenyl, hexenyl, octenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₁₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, heptynyl, and allenyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀); —CH═CH—CH═CH—; —CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxo, arylthioxo, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

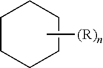

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

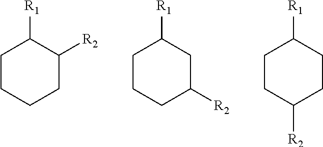

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R′″ and R″″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as an acetylfuran and a phenacyl group. Specific examples of acyl groups include acetyl and benzoyl.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, t-butoxyl, and n-pentoxyl, neopentoxy, n-hexoxy, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an amide group of the formula —CONH₂.

“Alkylcarbamoyl” refers to a R′RN—CO— group wherein one of R and R′ is hydrogen and the other of R and R′ is alkyl and/or substituted alkyl as previously described. “Dialkylcarbamoyl” refers to a R′RN—CO— group wherein each of R and R′ is independently alkyl and/or substituted alkyl as previously described.

The term carbonyldioxyl, as used herein, refers to a carbonate group of the formula —O—CO—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, iso-propylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —(C═O)— group.

The term “carboxyl” refers to the —COOH group. Such groups also are referred to herein as a “carboxylic acid” moiety.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R.

The term ureido refers to a urea group of the formula —NH—CO—NH₂.

Unless otherwise explicitly defined, a “substituent group,” as used herein, includes a functional group selected from one or more of the following moieties, which are defined herein:

(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and

(B) alkyl, heteroalkyl, cycloalkyl, heterocycloalkyl, aryl, and heteroaryl, substituted with at least one substituent selected from:

(i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and

(ii) alkyl, heteroalkyl, cycloalkyl, heterocycloalkyl, aryl, and heteroaryl, substituted with at least one substituent selected from:

(a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and

(b) alkyl, heteroalkyl, cycloalkyl, heterocycloalkyl, aryl, or heteroaryl, substituted with at least one substituent selected from oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “lower substituent” or “lower substituent group,” as used herein means a group selected from all of the substituents described hereinabove for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

Throughout the specification and claims, a given chemical formula or name shall encompass all tautomers, congeners, and optical- and stereoisomers, as well as racemic mixtures where such isomers and mixtures exist.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present disclosure. The compounds of the present disclosure do not include those which are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds that differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, zinc, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The term “pharmaceutically acceptable salts” is meant to include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like {see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in an acidic solution or otherwise contacted with an acid.

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)— catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

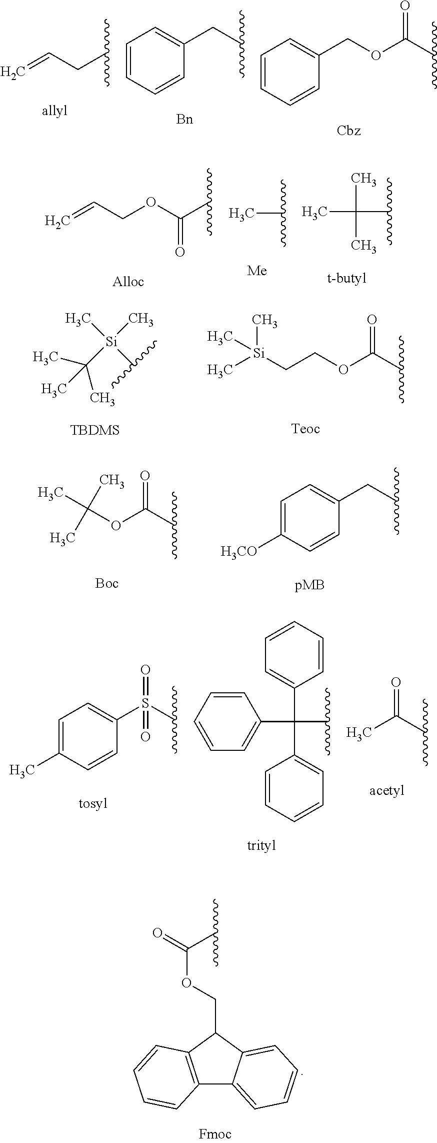

The term “subject” refers to an organism, tissue, or cell. A subject can include a human subject for medical purposes, such as diagnosis and/or treatment of an existing condition or disease or the prophylactic treatment for preventing the onset of a condition or disease, or an animal subject for medical, veterinary purposes, or developmental purposes. A subject also can include sample material from tissue culture, cell culture, organ replication, stem cell production and the like. Suitable animal subjects include mammals and avians. The term “mammal” as used herein includes, but is not limited to, primates, e.g, humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. The term “avian” as used herein includes, but is not limited to, chickens, ducks, geese, quail, turkeys, and pheasants. Preferably, the subject is a mammal or a mammalian cell. More preferably, the subject is a human or a human cell. Human subjects include, but are not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. A subject also can refer to cells or collections of cells in laboratory or bioprocessing culture in tests for viability, differentiation, marker production, expression, and the like.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

Referring now to FIGS. 1A-1D, it is shown that: (A) selective immunohistochemical labeling of cough receptor terminations in the guinea pig tracheal mucosa with antisera to the alpha3 subunit of Na+K+-ATPase; (B) the cough receptor terminals innerating the tracheal mucosa also are brilliantly and selectively labeled intravitally by the styryl dye FM2-10. Styryl dyes are known to produce fluorescence intensity proportional to Na+-K+-ATPase activity. Consistent with this notion, FM2-10 labeling of the cough receptor terminations is prevented by the sodium pump inhibitor ouabain (not shown). The data suggest that the cough receptors uniquely express the alpha3 subunit of the sodium pump and that it is highly active at baseline in these terminals (as evidenced by FM2-10 labeling); (C) the alpha3 subunit is known to be more sensitive to inhibition by ouabain than the housekeeping sodium pump utilizing alpha1 subunits. In functional studies, it was found that ouabain nearly abolished citric acid evoked coughing in anesthetized guinea pigs at a dose (30 μg/kg lv) that had no effect on heart rate, blood pressure or respiratory pattern at eupnea; (D) the benzimidazole H+-K+-ATPase inhibitor omeprazole, upon exposure to acid, is converted to a sulfenamide. The H+-K+-ATPase is expressed in the stomach, but not in the airways. The sulfenamides bind covalently and selectively to the alpha3 subunit of the sodium pump. Despite the lack of expression of the proton pumps in the airways (but likely because of the expression of the alpha3 subunit on the peripheral terminals of the cough receptors), topically applied omeprazole (0.1 mM) nearly abolished citric acid evoked coughing in anesthetized guinea pigs (FIG. 1).

### Example 2

Referring now to FIG. 2, the presently disclosed subject matter demonstrates that administration of nebulized acid-activated omeprazole (1 mg/mL), delivered as an aerosol to the airways, markedly inhibits citric acid evoked cough in conscious (unanesthetized) guinea pigs.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Canning, B. J., et al. (2004) Demonstration of the essential role of the alpha3-expressing isozyme of the Na+-K+-ATPase in regulating cough receptor activation in guinea pigs. Am. J. Respir. Crit. Care Med. 169(7):A799 (abstract).
Canning, B. J. (2006) Anatomy and neurophysiology of the cough reflex: ACCP evidence-based clinical practice guidelines. Chest 129 (1 Suppl):33S-47S.
Canning, B. J. and Mazzone S. B. (2005). Afferent pathways regulating the cough reflex. In Acute and Chronic Cough, A. B. Redington and A. H. Morice, eds. Taylor and Francis, Boca Raton, 24-48.
Canning, B. J. (2008) The cough reflex in animals: relevance to human cough research. Lung 186 Suppl 1:S23-8.
Canning B J (2007) Encoding of the cough reflex. Pulm Pharmacol Ther 20:396-401.
Canning B J, Farmer D G, Mori N (2006) Mechanistic studies of acid-evoked coughing in anesthetized guinea pigs. Am J. Physiol Regul Integr Comp Physiol 291(2):R454-R463.
Canning B J, Mazzone S B, Meeker S N, Mori. N, Reynolds S M, Undem B J (2004) Identification of the tracheal and laryngeal afferent neurones mediating cough in anaesthetised guinea-pigs. J Physiol 557 (Pt. 2):543-558.
Canning B J, Mori N, Mazzone S B (2006) Vagal afferent nerves regulating the cough reflex. Respir Physiol Neurobiol 1520:223-242.
de Carvalho A P, Sweadner K J, Penniston J T, Zaremba J, Liu L, Caton M, Linazasoro G, Borg M, Tijssen M A, Bressman S B, Dobyns W B, Brashear A, Ozelius L J (2004) Mutations in the Na+/K+-ATPase alpha3 gene ATP1A3 are associated with rapid-onset dystonia parkinsonism. Neuron 43:169-175.
Ebihara, S., et al. (2007) Contribution of gastric acid in elderly nursing home patients with cough reflex hypersensitivity. J Am Geriatr Soc. 55(10):1686-8.
Farley, R. A. (1984) The amino acid sequence of a fluorescein-labeled peptide from the active site of (Na,K)-ATPase. Biol. Chem. 259(15):9532-5.
Foley, T. D. (1997) 5-HPETE is a potent inhibitor of neuronal Na+,K(+)-ATPase activity. Biochem. Biophys Res. Commun. 235(2):374-6.
Irwin, R. S. (2006) Chronic cough due to gastroesophageal reflux disease: ACCP evidence-based clinical practice guidelines. Chest 129 (1 Suppl):80S-94S.
Irwin, R. S., et al. (2006) Diagnosis and management of cough executive summary: ACCP evidence-based clinical practice guideline. Chest 129(1 Suppl):1S-23S.
Iwata, H, et al. (1988) Difference between two isozymes of (Na++K+)-ATPase in interaction with omeprazole. Jpn J. Pharmacol 46(1):35-42.
Keeling, D J., et al. (1985) Studies on the mechanism of action of omeprazole, Biochem Pharmaco. 34(16):2967-73.
Matsuda, T., et al. (1984) Specific inactivation of alpha (+) molecular form of (Na++K+)-ATPase by pyrithiamin. J. Biol. Chem. 259(6):3858-63.
Matsuda T., et al. (1985) Involvement of sulfhydryl groups in the inhibition of brain (Na++K+)-ATPase by pyrithiamin, Biochim. Biophys. Acta. 817(1):17-24.
Mazzone, S. B, et al. (2009) Selective expression of a sodium pump isozyme by cough receptors and evidence for its essential role in regulating cough. J. Neurosci. 29(43):13662-71.
Morice, A. H. et al. (2004) The diagnosis and management of chronic cough, Eur. Respir. J. 24(3):481-92.
Oribe. Y., et al. (2005) Attenuating effect of H+K+ATPase inhibitors on airway cough hypersensitivity induced by allergic airway inflammation in guinea-pigs. Clin. Exp. Allergy 35(3):262-7,
Reifenberger, M. S., et al. (2008) The reactive nitrogen species peroxynitrite is a potent inhibitor of renal Na-K-ATPase activity. Am. J. Physiol. Renal Physiol. 295(4):F1191-8.
Sen, P. C., Pfeiffer, D. R. (1982) Characterization of partially purified (Na++K+)-ATPase from porcine lens. Biochim Biophys Acta 693(1); 34-44.
Siems, W. G., et al. (1996) 4-hydroxynonenal inhibits Na(+)-K(+)-ATPase. Free Radic Biol. Med. 20(2):215-23.
Swarts, H. G., et al. (1990) Binding of unsaturated fatty acids to Na+,K(+)-ATPase leading to inhibition and inactivation. Biochim Biophys Acta 1024(1):32-40.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for treating or preventing a cough in a subject in need of treatment thereof by modulating an activity of an alpha3 subunit of Na⁺K⁺-ATPase on one or more vagal afferent nerves of the subject, the method comprising:
(a) converting an acid-labile sulfenamide prodrug to a sulfenamide; and
(b) topically administering a therapeutically effective amount of the sulfenamide to the subject; whereby the sulfenamide modulates the activity of an alpha3 subunit of Na⁺K⁺-ATPase on one or more vagal afferent nerves of the subject to treat or prevent the cough.

2. The method of claim 1, wherein the converting of the acid-labile sulfenamide prodrug to a sulfenamide comprises contacting the prodrug with an acid.

3. The method of claim 1, wherein the acid-labile sulfenamide prodrug is selected from the group consisting of a benzimidazole and an imidazopyridine, and derivatives, enantiomers, isomers, tautomers, free bases, polymorphs, esters, hydrates, or salts thereof.

4. The method of claim 3, wherein the benzimidazole or imidazopyridine comprises a compound of formula (I): 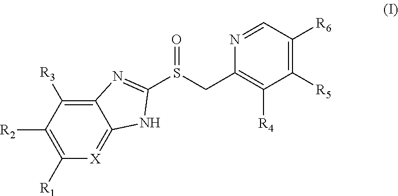
wherein:
X is CH or N;
R₁, R₂, and R₃ are the same or different and are selected from the group consisting of hydrogen, substituted and unsubstituted alkyl, alkoxyl, halogen, haloalkoxyl, alkylcarbonyl, alkoxycarbonyl, oxazolinyl, trifluoroalkyl, a heterocyclic ring that may be further substituted or adjacent groups R₁, R₂, and R₃ form ring structures, which may be further substituted;
R₄, R₅, and R₆ are the same or different and are selected from the group consisting of hydrogen, substituted or unsubstituted alkyl, alkoxyl optionally substituted by halogen, alkylthio, alkoxyalkoxyl, dialkylamino, piperidino, morpholino, halogen, phenyl and phenylalkoxyl;
and a derivative, enantiomer, isomer, tautomer, free base, polymoph, ester, hydrate, or salt thereof.

5. The method of claim 4, wherein the benzimidazole or imidazopyridine is selected from the group consisting of omeprazole, esomeprazole, hydroxyomeprazole, rabeprazole, pantoprazole, lansoprazole, s-lansoprazole, dexlansoprazole, pariprazole, ilaprazole, dontoprazole, habeprazole, perprazole, ransoprazole, nepaprazole, leminoprazole, tenatoprazole, and s-tenatoprazole.

6. The method of claim 4, wherein the sulfenamide has the following formula: 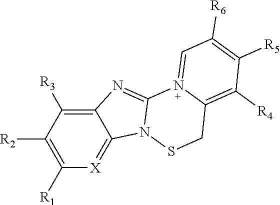
wherein R₁, R₂, R₃, R₄, R₅, R₆, and R₇ are as defined hereinabove.

7. The method of claim 1, wherein the sulfenamide is topically administered to the subject in an aerosol form.

8. The method of claim 7, wherein the sulfenamide is topically administered to one or more airways of the subject.

9. The method of claim 8, wherein the one or more airways of the subject is selected from the group consisting of the larynx, trachea, bronchi, and combinations thereof.

10. The method of claim 1, further comprising administering one or more zinc (Zn++) supplements in combination with the sulfenamide.

11. The method of claim 10, wherein the zinc supplement is selected from the group consisting of zinc gluconate, zinc sulfate, zinc acetate, and zinc citrate.

12. The method of claim 1, wherein the cough is selected from the group consisting of an acute cough and a chronic cough.

13. A method for identifying at least one candidate compound for treating or preventing a cough in a subject by modulating an activity of an alpha3 subunit of Na⁺K⁺ATPase on one or more vagal afferent nerves of the subject, the method comprising:
(a) obtaining a sample containing a functional alpha3 subunit of Na+K+ATPase on a vagal afferent nerve;
(b) contacting the sample with an amount of a candidate compound sufficient to modulate the activity of the alpha3 subunit of Na⁺K⁺ATPase on the vagal afferent nerve;
(c) measuring the activity of the alpha3 subunit of Na⁺K⁺ATPase on the vagal afferent nerve; and
(d) comparing the activity of the alpha3 subunit of Na⁺K⁺ATPase on the vagal afferent nerve that was contacted with the candidate compound with the activity an alpha3 subunit of Na⁺K⁺ATPase on the vagal afferent nerve that was not contacted with the candidate compound, wherein a modulation of the activity of the alpha3 subunit of Na⁺K⁺ATPase on the vagal afferent nerve by the candidate compound identifies the compound as a candidate to treat or prevent a cough.

14. The method of claim 13 wherein the candidate compound is activated with an acid before contacting it with the alpha3 subunit of Na⁺K⁺ATPase on the vagal afferent nerve.

15. The method of claim 14, wherein the candidate compound is selected from the group consisting of a benzimidazole and an imidazopyridine, and derivatives, enantiomers, isomers, tautomers, free bases, polymorphs, esters, hydrates, or salts thereof.
